(19) United States
(12) Patent Application Publication
(10) Pub. No.: US 2025/0186813 A1
(43) Pub. Date: Jun. 12, 2025

(54) AIR FILTRATION DEVICE FOR PROTECTION

(71) Applicant: WERLAX INVEST, S.L., GIRONA (ES)

(72) Inventors: JULIA FERRER SOLER, BARCELONA (ES); SILVIA SABRIA MESTRAS, BARCELONA (ES)

(21) Appl. No.: 18/840,890

(22) PCT Filed: Feb. 25, 2022
(86) PCT No.: PCT/ES2022/070104

§ 371 Date: Aug. 22, 2024

(51) Int. Cl. A62B 18/04; A62B 18/08

(52) U.S. Cl. CPC A62B 18/045; A62B 18/08

(57) ABSTRACT

An air filtration device for protection including a securing structure for securing to a user's head; a visor associated with the securing structure; a hood fixed thereto defining a closed chamber; a rear air inlet comprising an inlet filter; an air outlet; and a fan for generating an air flow from the air inlet through an upper air duct and through a front portion of the closed chamber to the air outlet. The air outlet is arranged on one side and comprises an outlet filter, a side frame and a side cover. The outlet filter is fixed between the side frame and the side cover.

### CROSS-REFERENCE TO RELATED APPLICATION

This is a National Stage application of PCT International Application PCT/ES2022/070104, filed on Feb. 25, 2022, which is incorporated herein by reference in its entirety.

### TECHNICAL FIELD

The present invention relates to an air filtration device for protection, of the type called Personal Protective Equipment (PPE).

### BACKGROUND

Nowadays, workers from different sectors commonly use Personal Protective Equipment (PPE) with the aim of protecting themselves against one or more risks that may threaten their safety or health.

One of these sectors is the health sector, where workers (doctors, nurses, assistants, etc.) are in direct contact with sick patients who are able to transmit their disease through infectious agents (airborne agents, fluids, etc.) to the healthcare worker or other patients. Moreover, there is the possibility that the healthcare worker him or herself will contract a disease (for example, in the family environment) and infect patients.

To prevent these risks, one of the PPEs most used by healthcare workers are masks (for example, those known as surgical masks), which consist of pieces of fabric or paper made of insulating material that is placed over the nose and mouth and secured with a band or strap on the head to prevent the transfer of infectious agents from the patient to the user or vice versa.

These masks have the significant drawback that they do not completely isolate the user, who runs the risk of getting sick from contact with infectious agents (for example, through the eyes, the skin, etc.), as well as the fact that the professional's own infectious agents (exhaled air, sweat, etc.) may endanger the patient's health.

Additionally, other PPEs, commonly called full face masks, are often used which cover a large part of the user's face. These full face masks comprise a transparent visor that is secured to the head by means of straps, covering the user's face and isolating it from the outside. Said full face masks usually comprise, in the lower portion thereof, a hole for the inlet and outlet of air which, in turn, comprises an interchangeable filter for filtering the air that passes through said hole. The main drawback of full face masks of this type is that it is very common or the visor to fog up due to the condensation of the air exhaled by the user, making it difficult to perform tasks. This fogging of the visor can lead to very dangerous situations such as, for example, if it occurs during an operation, where any mistake can endanger the life of the patient.

Another drawback of these full face masks is that they do not isolate the user's entire head, leaving the user exposed to infectious agents that can enter his or her body through the skin, or they can expose patients to the user's infectious agents, putting their health at risk.

Additionally, the use of PPEs commonly called respiratory protection hoods is known, PPEs which are comprised of a hood made of insulating material and a transparent visor which define a closed chamber that surrounds the user's entire head. These hoods usually comprise a hole in the rear portion thereof connected to a tube that leads to an external device which generates an air flow inside the hood (for example, an oxygen cylinder).

One of the drawbacks of these respiratory protection hoods is that said tube and external device make it difficult for the user to move (for example, if the tube catches on furniture, if the external device is an oxygen cylinder, etc.), which makes it difficult to carry out their work.

Another drawback of hoods of this type is that the air outlet is usually a hole arranged in the front portion of the hood, in a section close to the user's mouth. The surface of said front portion is occupied almost entirely by the visor, so there is not enough surface to house a filter with the necessary surface for filtering the air that passes through said hole. Therefore, these known hoods are not designed to prevent the user from infecting a patient.

Therefore, an air filtration device is needed which facilitates the user's freedom of movement and which isolates both the user and the patients from infectious agents.

### SUMMARY

In order to meet this objective and solve the technical problems discussed so far, in addition to providing additional advantages that may be derived later, the present invention provides an air filtration device for protection comprising:
a securing structure configured to be secured to a user's head;
a visor associated with the securing structure;
a hood associated with the securing structure, said hood defining a closed chamber after securing the securing structure to the user's head;
a rear air inlet comprising an inlet filter;
an air outlet; and
a fan configured to generate an air flow from the air inlet through an upper air duct and through a front portion of the closed chamber to the air outlet.

The air outlet is arranged on at least one side and comprises an outlet filter, a side frame and a side cover. The side cover has at least one air passage opening and a curved surface that defines an air chamber between the outlet filter and the side cover. The side cover is configured to be fixed to the side frame, such that the outlet filter is fixed between the side frame and the side cover.

Rear, upper, side and front are understood as the different positions of the device relative to the user's head when the device is in its position of use. Therefore, the combination of the visor and hood defines a closed chamber around the user's head, isolating the user's head from infectious agents coming from the outside and vice versa.

Moreover, the air flow generated by the fan supplies filtered air from the outside to the user, protecting him or her from infectious agents that could be in the air. As such, the device is not related to heavy objects that may limit the user's freedom of movement.

Moreover, since the air flow is directed to the front portion of the closed chamber, the user is continuously ventilated properly.

Since the air flow flows through the front portion of the closed chamber, i.e., where the visor is located, the possibility that the visor fogs up due to condensation from the air exhaled by the user is reduced.

As indicated, the air outlet is arranged on at least one side of the device in a position of use thereof, preferably near at least one ear of the user. More preferably, the air outlet is provided on each of the two sides. In this way, the surface available for the air filter is doubled and a better distribution of the air flow around the user's face is obtained.

Therefore, it is possible to place an outlet filter with the necessary surface, for the proper functioning of the air filtration device, in the air outlet since the side of the device comprises a large surface not occupied by the visor. But moreover, the arrangement of the outlet filter on the side allows the outlet filter to have a longer useful life because, due to its location and larger surface, it accumulates a smaller amount of moisture from the air exhaled by the user, thus prolonging the duration of use of the outlet filters until they are saturated.

Likewise, it is envisaged that the side frame is fixed to the hood, although it is not ruled out that it is fixed to the securing structure.

Furthermore, since the outlet filter is fixed between the side frame and the side cover, the outlet filter is easily replaceable.

Additionally, the air chamber between the outlet filter and the side cover allows most of the outlet filter surface to be used for filtering the air that passes through the air outlet.

Preferably, the side frame comprises an outer surface that is provided with ribs on which the outlet filter is arranged. This allows a space to be left between the outlet filter and the outer surface of the side frame, so that most of the outlet filter is used for filtering the air that passes through the air outlet. Said ribs on the side frame facilitate the condensation of moisture in the air that passes through the air outlet, increasing the useful life of the outlet filter.

Preferably, the side cover comprises ribs on the curved surface where the outlet filter is arranged. Said ribs enable reducing the area of the outlet filter in contact with the curved surface of the side cover. Thus, most of the outlet filter is used for filtering air that passes through the air outlet. Likewise, these ribs on the side cover facilitate the condensation of moisture in the air that passes through the air outlet, which increases the useful life of the outlet filter.

Preferably, the upper air duct comprises a plurality of subducts for a homogeneous distribution of the air flow along the visor, avoiding discomfort to the user due to the concentration of the air flow in a single section of the user's face and preventing fogging of the visor.

Preferably, the inlet filter has a greater filtering capacity than the outlet filter. In this way, the necessary surface of the outlet filter is reduced for the proper functioning of the air filtration device. This is due to the fact that the size of the outlet filter is related to the flow rate of the air flow that passes through the outlet filter, i.e., the greater the flow rate of the air flow, the greater the required surface of the outlet filter; and through the outlet filter material, i.e., the greater the filtering capacity of the outlet filter material, the greater the required surface of the outlet filter. Likewise, the flow rate of the air flow is directly related to the surface of the inlet filter, i.e., the greater the surface of the inlet filter, the greater the flow rate of the air flow; and to the inlet filter material, i.e., the greater the filtering capacity of the inlet filter, the lower the flow rate of the air flow; and to the power of the fan, i.e., the greater the power of the fan, the greater the flow rate of the air flow.

Preferably, the device comprises an external power supply unit that can be fixed to the user's waist, improving the portability of the device.

Preferably, the device comprises sound amplification means which at least comprise a microphone, a headset and/or a loudspeaker to allow communication between the user and the outside. This ensures that the user can clearly hear sound from outside the closed chamber and/or vice versa. It is envisaged that the microphone and/or the loudspeaker are located outside the closed chamber and the headset and/or the microphone are inside the closed chamber. Preferably, the sound amplification means are configured to cancel the sound emitted by the air flow and/or the ambient sound from the outside, for example, by cancelling a specific frequency, which reduces the discomfort of the user, improving their hearing.

Preferably, the device comprises a control unit configured to regulate the speed of the fan, for example by means of an integrated algorithm, for turning on, turning off, power control and/or rotation of the fan motor. This allows for greater control of the air flow inside the closed chamber. For this, it is envisaged that the control unit comprises at least one processor.

Preferably, the device comprises at least one air flow characteristic sensor inside the closed chamber. The control unit is configured to receive information from the air flow characteristic sensor, in particular, to continuously monitor information received from the air flow characteristic sensor, and to contribute to the various functions of the control unit, such as adapting the fan speed.

Preferably, the air flow characteristic sensor is a flow rate sensor, or flow meter, which measures the flow rate at the inlet of air flow into the closed chamber.

The use of a CO₂ concentration sensor with the aim that the control unit prevents said concentration from exceeding a set value of CO₂ concentration is not ruled out.

Likewise, it is not ruled out that the air flow characteristic sensor is a pressure sensor, temperature sensor, etc., or a combination of all of them.

Preferably, the control unit is configured to maintain a maximum pressure difference with respect to the outside atmospheric pressure inside the closed chamber of up to 0.5 mBar. In this way, the control unit allows an air flow inlet rate to be supplied inside the closed chamber which is suitable for supplying the user with sufficient breathable air to carry out difficult tasks and, at the same time, prevent the pressure difference between the inside of the closed chamber and the outside atmospheric pressure from exceeding 0.5 mBar. Thanks to this feature, discomfort to the user is avoided.

Preferably, the device comprises identification means for unequivocally identifying the device by means of a unique digital tag processed by the control unit. This tag will contain a series of related information called attributes. These attributes can only be modified if a set of conditions is met. The identity of the device will therefore be defined by this unique tag with attributes such as the encoded name of the user to whom the device is assigned, the number of times the assigned person has worn the device, the number of hours the assigned person wears the device until he or she takes it off, whether the CO₂ level or air flow rate has exceeded a programmed limit, etc.

Preferably, the device comprises data encryption means for data processed by the control unit, in particular, by means of a cryptographic algorithm. Encryption can be performed using “blockchain” and/or “tokenisation” technology. Said information that provides the device with a digital identity allows, thanks to a cryptographic algorithm, for the secure registration of information relevant to the device in a blockchain network, facilitating the maintenance of the device, the cleaning thereof and a change of assignment to another user. Likewise, said relevant information may be an identity and some attributes (for example, username, battery status, hours of use, last use, last disinfection, etc.), which may be updated as the useful life of the device passes. In this way, said information is converted into non-fungible tokens (“NFTs”), which will be created in a “blockchain” and managed through a “Smart Contract”. Programming said “Smart Contract” will also allow the device to be assigned to a specific user, allowing only this user to start this device. This concept is equivalent to that of a unique programmable electronic key that is personalized for a period of time, increasing the user's attention to device maintenance and security.

Identifying several air filtration devices in a “blockchain” network will allow the “Smart Contract” programming to gather user information in order to have a comparison of the use of the device according to user profile, its location and the need to update materials or electronics with which new functionality and maintenance improvement processes are generated.

Preferably, the control unit comprises wireless internet connection means, in particular via WI-FI, Bluetooth® technology, Zigbee technology, Near-Field Communication (NFC) technology, GSM, as well as sending and receiving point-to-point information, such as “LoRa”, and/or technology associated with the Internet of Things (IoT), such as Sigfox, directly to a server or an intermediate node, more particularly, using a frequency of the ISM (Industrial, Scientific and Medical) radio band and a proprietary protocol.

Extracting data between periods of use of the device will allow statistical data to be extracted to improve the device in the future and add more functionalities such that another attribute of the device may be the update version number of the algorithm with new functionalities.

Likewise, an object of the present invention is a cleaning system for the air filtration device which comprises sterilization means for sterilizing the device by means of UV-C radiation. This ensures correct disinfection of the air filtration device between uses, projecting UV-C light in a safe way that cannot be harmful to humans.

Additionally, this cleaning can be combined with charging the external power supply unit. It is envisaged that the sterilization means may be in the form of a sleeve into which the device is inserted.

With the initial data from the deployment of use of the air filtration devices in a secure and anonymous manner, an accurate prediction of potential failures can be made in order to treat them and minimize any maintenance time.

The data transmitted securely in the “Blockchain” may be collected during several periods to visually represent the deployment data and use, allowing for a clear visual display of the deployment in order to encourage its use in certain locations.

The definition of the air filtration device as “NFT” will enable gathering feedback from each user in the future to improve their specific device and it will also enable different professionals to share suggestions on new functionalities of use and facilitate their acquisition and the possibilities of minimizing any negative impact on sustainability.

### DESCRIPTION OF THE DRAWINGS

A non-limiting example of the present invention is described below with reference to the drawings attached, wherein:

FIG. 1 shows a perspective view of a preferred exemplary embodiment of the air filtration device according to the present invention.

FIG. 2 shows an elevation view of the device of FIG. 1 with some internal portions of the same being visible.

FIG. 3 is an exploded perspective view of the air filtration device.

FIG. 4 are perspective views that show the steps to follow to correctly place the air filtration device.

FIG. 5 is a partial perspective view of the air inlet.

FIGS. 6A to 6F are perspective views that show the steps to follow to remove the inlet filter.

FIG. 7 shows a top view of the upper air duct where the air distribution subducts at the outlet of the air duct have been schematically illustrated.

FIG. 8 shows an exploded perspective view of the air outlet.

FIG. 9 shows a perspective view of the side cover.

### DETAILED DESCRIPTION OF THE INVENTION

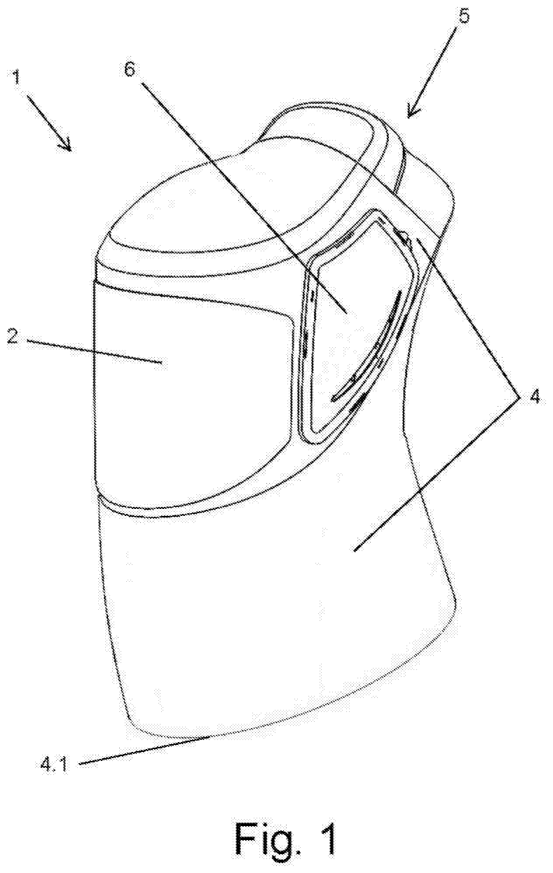

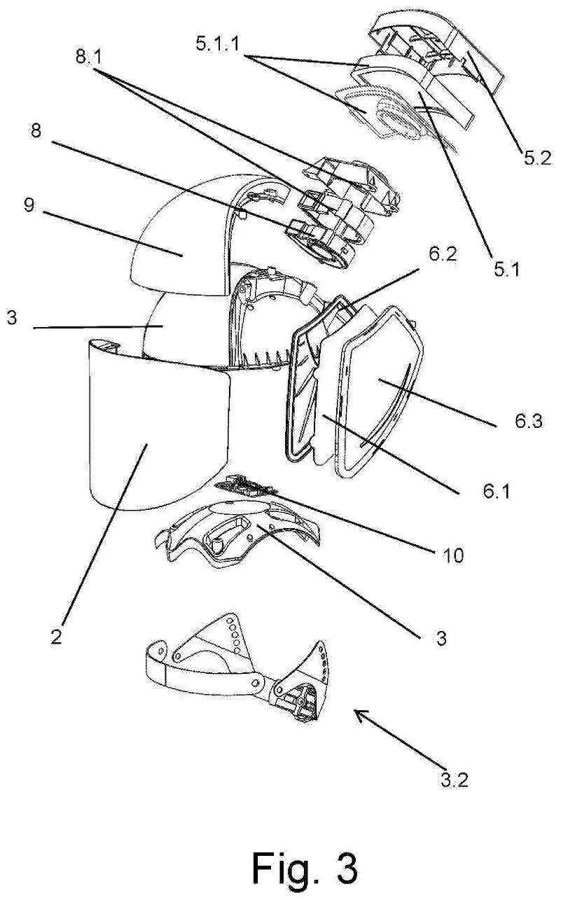

The non-limiting example illustrated in FIGS. 1-9 corresponds to an air filtration device 1. The air filtration device 1 comprises a transparent visor 1 to allow the user to see once the air filtration device 1 has been placed on the user's head.

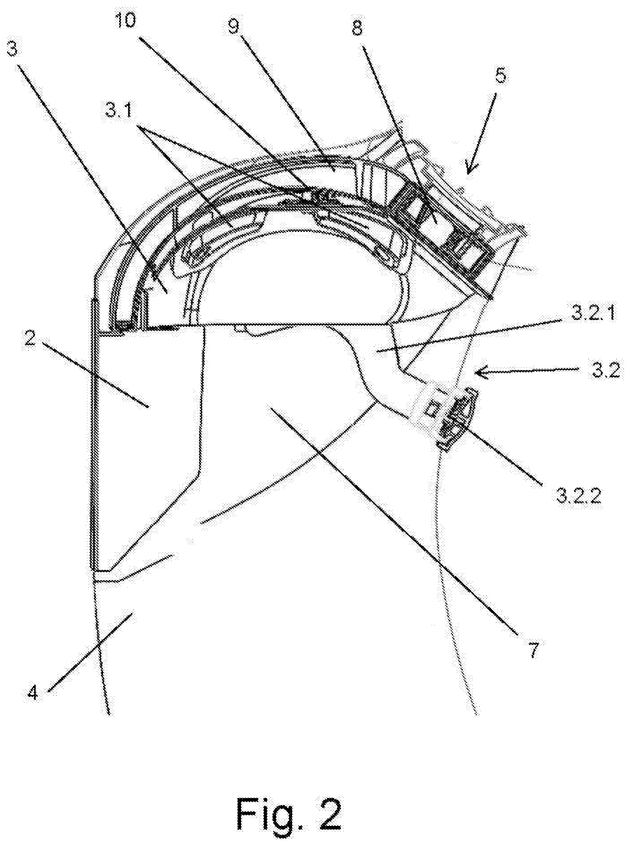

As can be seen in FIGS. 1 and 2, the visor 1 is attached to a securing structure 1. The securing structure 1 is configured to be secured to a user's head.

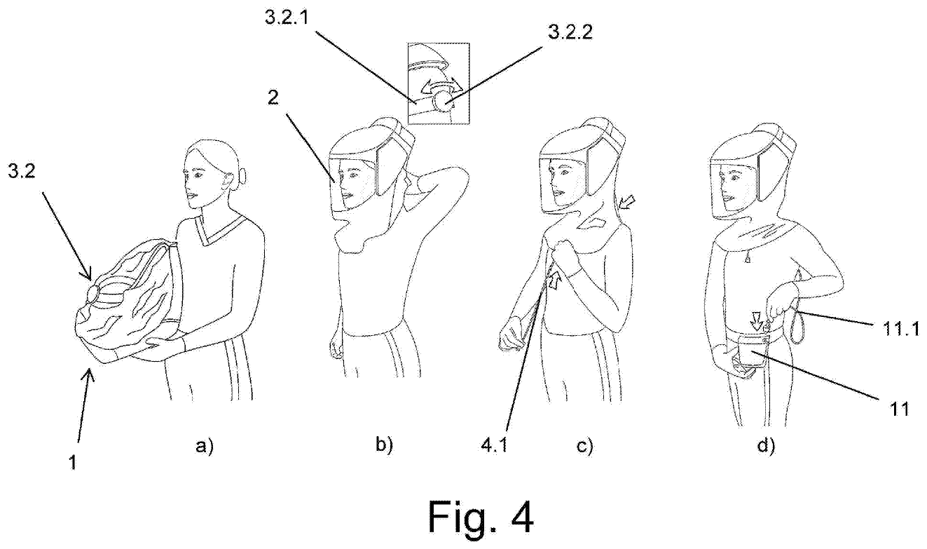

The visor 1 is arranged in such a way that, once the securing structure 1 is secured to the user's head, the visor 1 is in front of the user's face and at such a distance that it leaves an appropriate gap between the face and the visor 1, as shown in FIGS. 4c and 4d. Therefore, the possibilities that the visor 1 fogs up are limited and the discomfort that the visor 1 would cause if it were very close to the face is reduced.

The securing structure 1 comprises, as can be seen in FIG. 2, protrusions 3.1 to support the user's head. It is envisaged that these protrusions 3.1 may be cushioned to improve user comfort.

Likewise, the securing structure 1 comprises securing means 3.2. In the example shown, the securing means 3.2 comprise straps 3.2.1 and a fastening means 3.2.2. The fastening means 3.2.2 comprises means for adjusting the fastening force so that the user can properly secure the air filtration device 1 on his or her head.

The air filtration device 1 additionally comprises a hood 4 fixed both to the visor 1 and to the securing structure 1. When the securing structure 1 is secured to the user's head, the hood 4 surrounds the user's head and neck. Once the air filtration device 1 is secured to the user's head, the hood 4 and visor 1 define a closed chamber 7 around the user's head, as shown in FIG. 2.

Preferably, the hood 4 is made up of a material that prevents the passage of air, such as a material in accordance with standard UNE EN 13795, which prevents the passage of infectious agents from outside the closed chamber 7 to the inside and vice versa.

To ensure the airtightness of the closed chamber 7, the hood 4 comprises airtight closing means 4.1 (for example, a cord, tie, etc.) in the lower portion of the hood 4.

The lower portion of the hood 4 is understood as the section of the hood 4 that is at the lower end when the air filtration device 1 is secured to the user's head.

As can be seen in FIG. 2, the air filtration device 1 comprises a control unit 10. In the example shown, the control unit 10 is fixed to the securing structure 1.

In the example, an external unit 11 for supplying electric power to the control unit 10 is also provided. The external unit 11 comprises an electrical connection means 11.1 for electrically connecting the external unit 11 to the control unit 10.

It should be noted that although the use of an external unit 11 for supplying electric power is mentioned in this preferred exemplary embodiment, it is not ruled out that the air filtration device 1 comprises an electric battery inside the closed chamber 7 for supplying electric power to the control unit 10.

FIG. 4 shows the steps to follow to secure the air filtration device 1 to the user's head. These steps are:
a) securing the air filtration device 1, so that the securing structure 1 is not covered by the hood 4,
b) placing the securing structure 1 on the user's head so that the visor 1 is in front of the user's face and fastening the straps 3.2.1 on the back of the head by means of the fastening means 3.2.2;
c) making use of the closing means 4.1, in this case a cord, of the hood 4 ensuring the airtightness of the closed chamber 7; and
d) fixing the external unit 11 to the waist, connecting the external unit 11 to the air filtration device 1 by means of the electrical connection means 11.1.

The air filtration device 1 comprises an air inlet 5 to allow the passage of breathable air inside the closed chamber 7. Said air inlet 5 comprises, in turn, an inlet filter 5.1 for filtering the air that passes through said air inlet 5, so as to ensure that the air entering the closed chamber 7 is free of infectious agents.

In order to generate an air flow 12 from the outside of the air inlet 5 to the inside of the closed chamber 7, the air inlet 5 connects, on one side, to the outside of the closed chamber 7 and, on the other, to a fan 8, more specifically a centrifugal fan 8.

The fan 8 is configured to draw in air from outside the closed chamber 7 through the inlet filter 5.1. Likewise, the fan 8 is configured to introduce a flow rate, for example, of more than 120 l/min, inside an air duct 9. The air duct 9 is configured to distribute the flow rate of the air flow 12 to a front portion of the closed chamber 7.

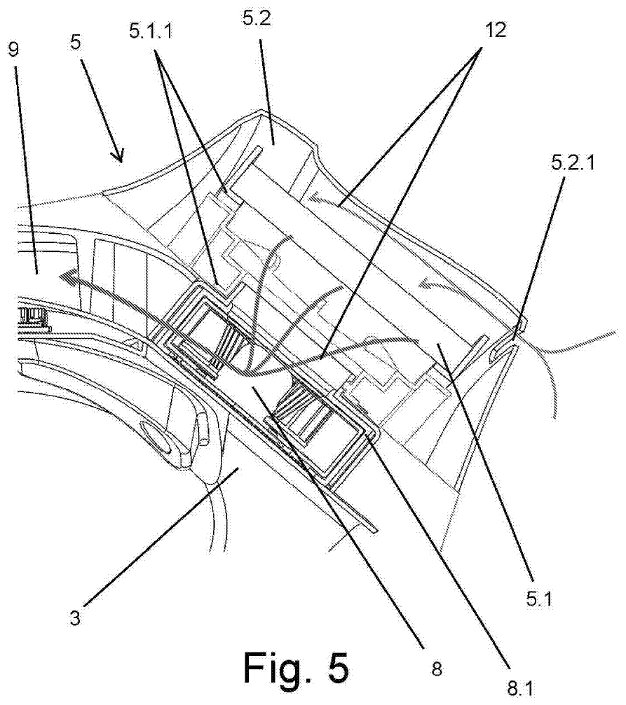

As can be seen in FIG. 5, the air inlet 5 comprises a covering 5.2 that protects the inlet filter 5.1 from splashes or blows that could affect the filtration capacity of the inlet filter 5.1. Moreover, the covering 5.2 comprises an inlet hole 5.2.1 through which air from the outside enters.

The inlet filter 5.1 is mounted inside an inlet frame 5.1.1. The inlet frame 5.1.1 can be fixed to a cavity 8.1 of the securing structure 1 where the fan 8 is arranged, by means of a bayonet fitting.

As can be seen in FIG. 5, the hood 4 remains fixed to the air inlet 5 by trapping between the inlet frame 5.1.1 and the cavity 8.1.

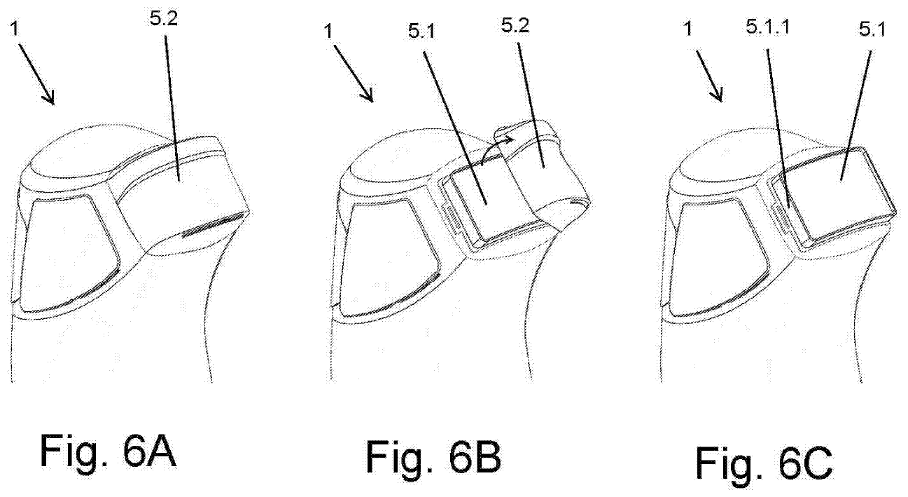

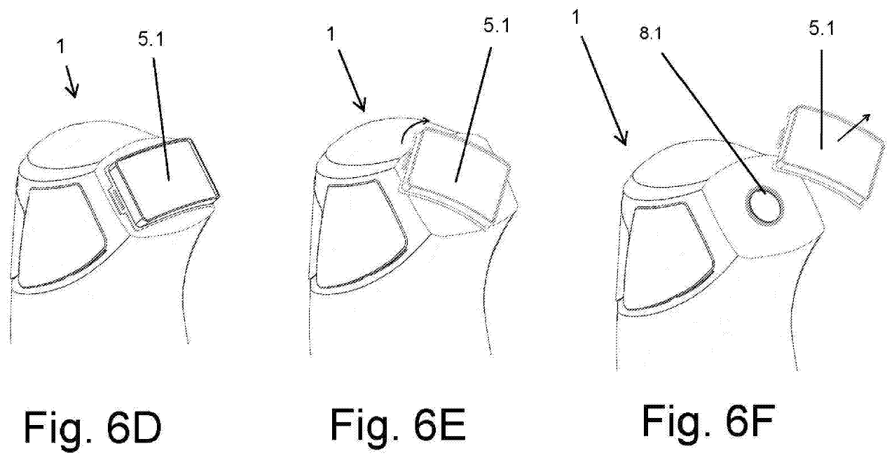

As can be seen in FIGS. 6A to 6F, the air inlet 5 is configured to be able to remove the inlet filter 5.1. To do this, firstly, the covering 5.2 is detached, as can be seen in FIGS. 6A to 6C, so that the inlet frame 5.1.1 is exposed. Next, and as can be seen in FIGS. 6D to 6F, the inlet frame 5.1.1 is rotated, detaching it from the fan 8 and it is separated from the air outlet 6. Finally, to place a new inlet filter 5.1 with the corresponding inlet frame 5.1.1 thereof, these steps are repeated in the opposite order.

As can be seen in FIG. 2, the air duct 9 is located in the upper portion of the air filtration device 1.

As mentioned previously, rear, upper, side and front are understood as the different positions of the device relative to the user's head when the air filtration device 1 is in its position of use.

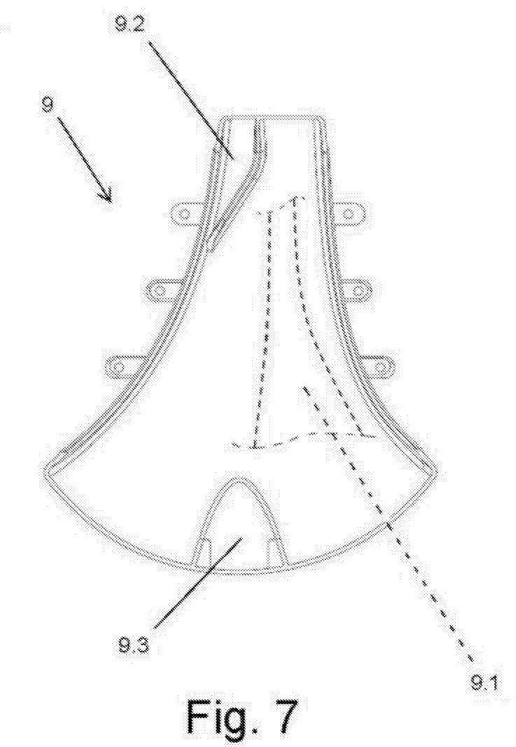

As can be seen in FIG. 7, the air duct 9 comprises several subducts 9.1 which have been illustrated schematically, such that their location and extension can vary depending on the design and the needs. The subducts 9.1 allow for a homogeneous distribution of the air flow 12 at the outlet of the air duct 9, which is the section opposite the fan 8. The outlet of the air duct 9 is arranged in a front portion of the closed chamber 7, more specifically in an upper portion of the visor 1.

The upper portion of the visor 1 is understood as the section of the visor 1 that is at the upper end when the air filtration device 1 is in its position of use.

It is also foreseen that the air duct 9 comprises an LED lighting means 9.3 in communication with the control unit 10. This LED lighting means 9.3 would be located in such a way that it is within the user's range of view when the air filtration device 1 is in its position of use.

In this way, the control unit 10 can inform the user of different statuses of the air filtration device 1 by means of a series of colors (for example, green if the status is optimal, orange if the external unit 11 has a low battery, etc.).

Likewise, the air duct 9 comprises a housing 9.2 to house an air flow characteristic sensor for the air flow entering the air duct 9.

Preferably, the air flow characteristic sensor is a flow meter, which will be in communication with the control unit 10, which receives information on the inlet flow rate of the air flow 12.

It is not ruled out that said air flow characteristic sensor is a CO₂ concentration sensor, a pressure sensor, a temperature sensor, etc., nor is it ruled out that the device comprises several air flow characteristic sensors.

With the information obtained from the air flow characteristic sensor, the control unit can control the fan 8 (turning it on, turning it off, changing the power and/or rotation speed of the motor, etc.) so as to maintain a maximum pressure difference with respect to the outside atmospheric pressure inside the closed chamber 7 of up to 0.5 mBar.

This pressure difference between the outside pressure (for example, 1 atmosphere, i.e., 1013.25 mBar) and the pressure inside the closed chamber 7 (in this example between 1013.25 mBar and 1013.75 mBar) allows the user to be properly ventilated.

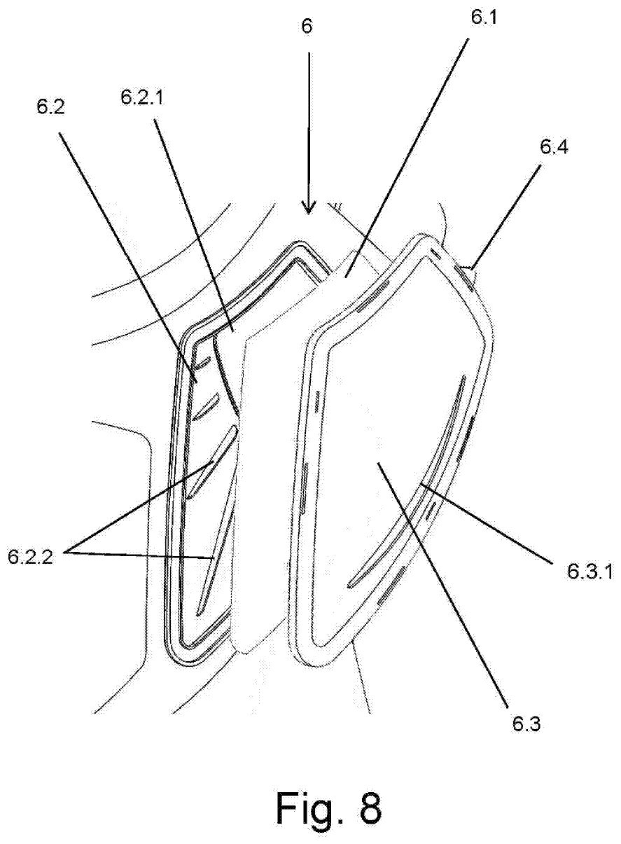

As can be seen in FIG. 8, the air outlet 6 is located on at least one side of the air filtration device 1 and comprises an outlet filter 6.1. This allows the user's infectious agents to be effectively filtered by the outlet filter 6.1 when leaving the closed chamber 7.

To ensure that the pressure difference in the closed chamber 7 is less than 0.5 mBar, the inlet filter 5.1 and the outlet filter 6.1 are sized according to the inlet flow rate of the air flow 12. In other words, the greater the flow rate, the greater the required surface of the inlet filter 5.1 and of the outlet filter 6.1.

In the non-limiting example shown in the drawings, the air filtration device 1 comprises two air outlets 6. The air outlets 6 can be the same or have different features. Each air outlet 6 comprises an outlet filter 6.1 and they are arranged on each side of the device 1. In this way, the effective surface for the outlet filter 6.1 is doubled and a better distribution of the air flow 12 through the visor 1 and around the user's face is obtained.

As shown in FIG. 8, each air outlet 6 comprises a side frame 6.2 fixed to the hood 4. Said side frame 6.2 comprises an outlet hole 6.2.1 for the outlet of air from inside the closed chamber 7.

Likewise, the side frame 6.2 comprises ribs 6.2.2 on an outer surface of the side frame 6.2. The ribs 6.2.2 allow the condensation of the moisture in the air that passes through the air outlet 6 to be absorbed. Moreover, the ribs 6.2.2 allow a space to be left between the outlet filter 6.1 and the outer surface of the side frame 6.2, such that most of the surface of the outlet filter 6.1 can be used to filter the air that passes through the air outlet 6.

In the non-limiting example shown in the drawings, a side cover 6.3 is arranged attached to the side frame 6.2 by snapping or clipping. The side cover 6.3 allows the outlet filter 6.1 to be protected from splashes or blows that could affect the filtration capacity of the outlet filter 6.1. The side cover 6.3 comprises an opening 6.3.1 for the passage of the air flow 12 through the air outlet 6. Both the side frame 6.2 and the side cover 6.3 comprise a flange 6.4 to make it easier to detach the side cover 6.3 when the outlet filter 6.1 must be replaced.

Likewise, the side cover 6.3 has a curved surface that defines an air chamber between the surface of the outlet filter 6.1 and the side cover 6.3. In this way, it ensures that the entire surface of the outlet filter 6.1 can be used for filtering the air that passes through the air outlet 6.

The side frame 6.2 comprises ribs 6.2.2 on the outer surface of the side frame 6.2. Said ribs 6.2.2 allow the condensation of the moisture in the air that passes through the air outlet 6 to be absorbed.

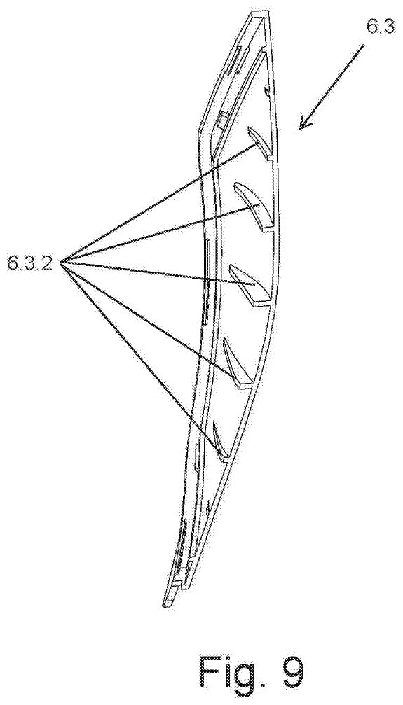

Additionally, the side cover 6.3 comprises ribs 6.3.2 on the curved surface, as can be seen in FIG. 9.

In a preferred exemplary embodiment, the outlet filter 6.1 is first arranged on the ribs 6.3.2 of the side cover 6.3, to then fix the side cover 6.3 to the side frame 6.2 without touching the outlet filter 6.1.

In another preferred exemplary embodiment, the outlet filter 6.1 is first arranged on the ribs 6.2.2 of the side frame 6.2, to then fix the side cover 6.3 to the side frame 6.2 without touching the outlet filter 6.

It is foreseen that the material of the inlet filter 5.1 has a filtering capacity according to the category P3 in accordance with standard UNE EN 143:2021, which specifies performance requirements and test methods for filters designed to filter particles (i.e., capable of filtering viruses, among other things). The air that is introduced inside the closed chamber 7 is highly filtered. This is very important in the event that the user is in contact with patients with very contagious diseases.

Likewise, it is foreseen that the material of the outlet filter 6.1 has a filtering capacity according to a type II category in accordance with standard UNE EN 14683:2019, which specifies performance requirements and test methods for filters designed to filter bacteria.

In this way, the inlet filter 5.1 will filter all the air that enters the closed chamber 7, protecting the user against particles and viruses from the outside, and the exhaled air will be filtered by the outlet filter 6.1, filtering bacteria, i.e., filtering the contribution that the user can make to the filtered air at the air inlet 5.

The air expelled through the air outlet 6 has previously been filtered by the inlet filter 5.1. This air only contains the user's infectious agents, so it is not necessary to use an outlet filter 6.1 with a filtering capacity in accordance with standard UNE EN 143:2021.

The material of the inlet filter 5.1 has a greater filtering capacity than the outlet filter 6.1. In this way, the necessary surface of the outlet filter 6.1 is reduced for the correct functioning of the air filtration device 1.

For example, in the event that the material of the outlet filter 6.1 was in accordance with the standard UNE EN 143:2021 (category P3) and the surface of the outlet filter 6.1 is not modified, a pressure difference between the outside and the inside of the closed chamber 7 of more than 0.5 mBar would be required, which would cause discomfort to the user. This would require increasing the surface of the outlet filter 6.1, and therefore of the air outlet 6 and of the air filtration device 1, which would result in discomfort for the user (for example, due to the increase in weight, volume, etc.).

It is also envisaged that the external unit 11 comprises loudspeakers so that the user's voice can be clearly heard outside the closed chamber 7.

Likewise, the possibility that the control unit 10 is arranged in the external unit 11 is envisaged.

It is also foreseen that the control unit 10 can be configured to perform the following tasks:
noise cancellation of the fan 8, thereby improving the user's hearing;
Bluetooth® communication for communication by telephone, use of a digital stethoscope, or for communication with the user's headphones;
increasing the volume of the loudspeaker of the external unit 11 for those patients with hearing problems;
analyzing the frequency of use of the air filtration device 1, the user's movements;
verifying the user's identity;
informing the user when it is necessary to replace the inlet filter 5.1 and/or the outlet filter 6.1, normally by means of the LED lighting means 9.3.

What is claimed is:

1. An air filtration device for protection comprising:
a securing structure configured to be secured to a user's head;
a visor associated with the securing structure;
a hood associated with the securing structure, said hood defining a closed chamber after securing the securing structure to the user's head;
a rear air inlet comprising an inlet filter;
an air outlet; and
a fan configured to generate an air flow from the air inlet through an upper air duct and through a front portion of the closed chamber to the outlet air;
wherein the air outlet is arranged on at least one side and comprises an outlet filter, a side frame and a side cover, the side cover having at least one air passage opening and a curved surface that defines an air chamber between the outlet filter and the side cover, the side cover being configured to be fixed to the side frame, such that the outlet filter is fixed between the side frame and the side cover.

2. The air filtration device according to claim 1, wherein the side frame comprises an outer surface that is provided with ribs on which the outlet filter is arranged.

3. The air filtration device according to claim 1, wherein the side cover comprises ribs on the curved surface where the outlet filter is arranged.

4. The air filtration device according to claim 1, wherein the upper air duct comprises a plurality of subducts for a homogeneous distribution of the air flow along the visor.

5. The air filtration device according to claim 1, wherein the inlet filter has a greater filtering capacity than the outlet filter.

6. The air filtration device according to claim 1, further comprising an external power supply unit that can be fixed to the user's waist.

7. The air filtration device according to claim 1, further comprising sound amplification means, said sound amplification means comprising at least a microphone, a headset and/or a loudspeaker to allow communication between the user and the outside.

8. The air filtration device according to claim 7, wherein the sound amplification means are configured to cancel the sound emitted by the air flow and/or the ambient sound from the outside.

9. The air filtration device according to claim 1, further comprising a control unit configured to regulate the speed of the fan.

10. The air filtration device according to claim 9, further comprising at least one air flow characteristic sensor inside the closed chamber, the control unit being configured to receive information from the air flow characteristic sensor, in particular, to continuously monitor the information received from the air flow characteristic sensor.

11. The air filtration device according to claim 10, wherein the control unit is configured to maintain a maximum pressure difference with respect to the outer atmospheric pressure inside the closed chamber of up to 0.5 mBar.

12. The air filtration device according to claim 11, further comprising identification means for unequivocally identifying the device by means of a unique digital tag processed by the control unit.

13. The air filtration device according to claim 12, further comprising data encryption means for data processed by the control unit, in particular, by means of a cryptographic algorithm, more particularly, the encryption being performed using “blockchain” and/or “tokenisation” technology.

14. The air filtration device according to claim 13, wherein the control unit comprises wireless internet connection means, in particular via WI-FI, Bluetooth®, Zigbee, NFC, GSM, LoRa and/or Sigfox, directly to a server or to an intermediate node, more particularly, using a frequency of the ISM band and a proprietary protocol.

15. A cleaning system for an air filtration device according to claim 1, wherein the cleaning system comprises sterilization means for sterilizing the device by means of UV-C radiation.
